(19) United States
(12) Patent Application Publication
(10) Pub. No.: US 2008/0114177 A1
(43) Pub. Date: May 15, 2008

(54) METHOD FOR PREPARING FLUORINATED BENZOTRIAZOLE COMPOUNDS

(75) Inventors: Larry D. BOARDMAN, Woodbury, MN (US); George W. Griesgraber, Eagan, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/560,078

(22) Filed: Nov. 15, 2006

(51) Int. Cl. C07D 249/18

(52) U.S. Cl. 548/261

(57) ABSTRACT

Fluorinated benzotriazole compounds may be prepared by contacting a fluorochemical monofunctional compound, such a fluorinated alcohol, with a carboxybenzotriazole in the presence of a coupling agent, and an optional amine catalyst.

### FIELD OF THE INVENTION

The present invention describes a method for preparing fluorinated benzotriazole compounds.

### BACKGROUND

Fluorinated benzotriazole esters have been recently described in the treatment of metal and metal oxide surfaces to impart release, antisoiling, antistaining, repellency, hydrophobicity, oleophobicity and/or cleanability properties thereto.

U.S. Pat. No. 6,376,065 (Korba et al.) describes fluoroalkyl benzotriazoles that chemically bond to metal and metalloid surfaces and provide, for example, release characteristics to those surfaces. The compounds are characterized as having a benzotriazole head group which bonds to a metallic or metalloid surface and a fluoroalkyl tail portion. The compounds of the invention when applied to a metallic or metalloid surface form durable, self-assembled films that are monolayers or substantially monolayers.

U.S. Pub. No. 2005/0166791 (Flynn et al.) describes perfluoropolyether benzotriazole compounds that can be attached to a substrate having a metal or metal oxide-containing surface to provide at least one of the following characteristics: anti-soiling, anti-staining, ease of cleaning, repellency, hydrophobicity, or oleophobicity.

However, currently described methods of preparing fluorinated benzotriazole esters are less than satisfactory. U.S. Pat. No. 6,376,065 (Korba et al.) describes a trifluoroacetic acid catalyzed condensation of a fluoroalcohol with a carboxybenzotriazole to produce the described esters. The described method has been observed to suffer from low or variable yields, considerable byproduct formation and difficult separation of the desired product from byproducts.

### SUMMARY

The present method overcomes the deficiencies of prior art methods by providing a method that is amendable to the preparation of a variety of fluorinated benzotriazole esters and provides good yields and simpler separations. The method comprises contacting a fluorochemical monofunctional compound, such a fluorinated alcohol, with a carboxybenzotriazole in the presence of a coupling agent, and an optional amine catalyst.

Fluorinated benzotriazole esters of Formula I may be prepared by the method of the invention.

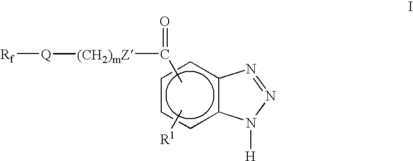

wherein R_{f}is a perfkuoroalkyl or a perfluoroheteroalkyl group, Z′ is —O—, —S—, or —NR⁵—, where R⁵ is H or C₁-C₄ alkyl, R¹ is H, a C₁-C₆ alkyl, or R_{f}-Q-(CH₂)ₘ-Z′-C(O)—, Q is a covalent bond or an organic linking group selected from a sulfonamido group, a carboxamido group, a carboxyl group, or a sulfonyl group, and m is at least 1.

Useful benzotriazole starting materials include those of the formula II

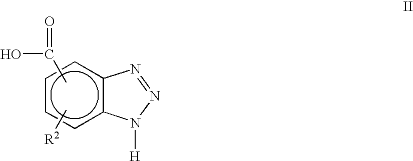

where the indicated carboxyl group is at the 5- or 6-position of the benzotriazole, and R² is H, a C₁-C₆ alkyl, or —CO₂H, which may be in any of the remaining 4-, 5-, 6- or 7-position of the benzotriazole. Preferably Formula I is a 5-carboxy, 6-carboxy, or 5-,6-dicarboxy benzotriazole.

“Fluorochemical monofunctional compound” means a compound having one nucleophilic functional group (such as a hydroxyl, primary or secondary amino or thio) and a perfluoroalkyl or a perfluoroheteroalkyl group (including perfluoropolyethers), e.g. C₄F₉SO₂N(CH₃)CH₂CH₂OH, C₄F₉SO₂N(CH₃)CH₂CH₂NH₂, C₄F₉CH₂CH₂OH, C₄F₉CH₂CH₂SH, C₂F₅O(C₂F₄O)₃CF₂CONHC₂H₄OH, C₆F₁₃CH₂OH, C₆F₁₃CH₂N(CH₃)OH, C₄F₉(CH₂)₁₁OH and the like.

“Fluorinated benzotriazole compound” means a compound derived from the reaction of at least one carboxybenzotriazole compound and one or more fluorochemical monofunctional compounds.

“Perfluoroalkyl” means that all or essentially all of the hydrogen atoms of the alkyl radical are replaced by fluorine atoms and the number of carbon atoms is from 2 to about 12, e.g. perfluoropropyl, perfluorobutyl, perfluorooctyl, and the like.

“Perfluoroalkylene” means that all or essentially all of the hydrogen atoms of the alkylene radical are replaced by fluorine atoms, e.g., perfluoropropylene, perfluorobutylene, perfluorooctylene, and the like

“Perfluoroheteroalkyl” means that all or essentially all of the hydrogen atoms of the heteroalkyl radical are replaced by fluorine atoms and the number of carbon atoms is from 3 to about 100, e.g. CF₃CF₂OCF₂CF₂—, CF₃CF₂O(CF₂CF₂O)₃CF₂CF₂—, C₃F₇O(CF(CF₃)CF₂O)ₚCF(CF₃)CF₂— where p is, for example, from about 3 to about 25, and the like.

“Perfluoroheteroalkylene” means that all or essentially all of the hydrogen atoms of the heteroalkylene radical are replaced by fluorine atoms, and the number of carbon atoms is from 3 to about 100, e.g. —CF₂OCF₂—, —CF₂O(CF₂O)ₐ(CF₂CF₂O)_{b}CF₂—, and the like, where a and b are arbitrary numbers for illustrative purposes.

Fluorochemical monofunctional compounds, useful in preparing the fluorinated benzotriazole compounds include those that comprise at least one R_{f}group and one nucleophilic functional group. The R_{f} groups can contain straight chain, branched chain, or cyclic fluorinated alkylene groups or any combination thereof. The R_{f} groups can optionally contain one or more heteroatoms (i.e. oxygen, sulfur, and/or nitrogen) in the carbon-carbon chain so as to form a carbon-heteroatom carbon chain (i.e. a heteroalkylene group). Fully-fluorinated groups are generally preferred, but hydrogen or chlorine atoms can also be present as substituents, provided that no more than one atom of either is present for every two carbon atoms. It is additionally preferred that any R_{f}group contain at least about 40% fluorine by weight, more preferably at least about 50% fluorine by weight. The terminal portion of the group is generally fully-fluorinated, preferably containing at least three fluorine atoms, e.g., CF₃O—, CF₃CF₂—, CF₃CF₂CF₂—, CF₃CF₂CF₂CF₂—, (CF₃)₂N—, (CF₃)₂CF—, SF₅CF₂—. In some embodiments, perfluoroalkyl groups (i.e., those of the formula CₙF₂ₙ₊₁— wherein n is 1 to 22 inclusive) are the preferred R_{f} groups, with n=3 to 5 being more preferred.

Useful perfluoroheteroalkyl groups correspond to the formula:

R_{f}¹—O—R_{f}²—(R_{f}³)_{q}— (III)

wherein R_{f}¹ represents a perfluorinated alkyl group, R_{f}² represents a perfluorinated polyalkyleneoxy group consisting of perfluoroalkyleneoxy groups having 1 to 4 carbon atoms or a mixture of such perfluoroalkyleneoxy groups, R_{f}³ represents a perfluoroalkylene group and q is 0 or 1. The perfluoroalkyl group R_{f}¹ in formula (III) may be linear or branched and may comprise 1 to 10 carbon atoms, preferably 1 to 6 carbon atoms. A typical perfluorinated alkyl group is CF₃—CF₂—CF₂—. R_{f}³ is a linear or branched perfluorinated alkylene group that will typically have 1 to 6 carbon atoms. For example, R_{f}³ is —CF₂—or —CF(CF₃)—. Examples of perfluoroalkyleneoxy groups of perfluorinated polyalkyleneoxy group R_{f}² include:
—CF₂—CF₂O—, —CF(CF₃)—CF₂O—, —CF₂—CF(CF₃)—O—, —CF₂—CF₂—CF₂O—, —CF₂O—, —CF(CF₃)—O—, and/or —CF₂—CF₂—CF₂—CF₂—O—.

The perfluoroalkyleneoxy group may be comprised of the same perfluoroalkyleneoxy units or of a mixture of different perfluoroalkyleneoxy units. When the perfluoroalkyleneoxy group is composed of different perfluoroalkyleneoxy units, they can be present in a random configuration, alternating configuration or they can be present as blocks. Typical examples of perfluorinated poly(perfluoroalkyleneoxy) groups include:
—[CF₂—CF₂—O]ᵣ—; —[CF(CF₃)—CF₂—O]ₛ—; —[CF₂CF₂—O]ₜ—[CF₂O]ᵤ— and
—[CF₂—CF₂—O]ᵥ—[CF(CF₃)—CF₂—O]_{w}—; wherein r is an integer of 2 to 25, s, t, u, v and w are independently integers of 3 to 25. A preferred perfluorinated polyether group that corresponds to formula (III) is CF₃—CF₂—CF₂—O—[CF(CF₃)—CF₂O]ₛ—CF(CF₃)— wherein s is an integer of 3 to 25. Such perfluorinated polyether groups are preferred in particular because of their benign environmental properties.

Useful fluorochemical monofunctional compounds include those of the following formula IV:

R_{f}-Q-(CH₂)ₘZ (IV)

wherein:
R_{f}is a perfluoroalkyl group having 1 to 22 carbon atoms, or a perfluoroheteroalkyl group having 3 to about 50 carbon atoms with all perfluorocarbon chains (perfluoroalkyl and perfluoroalkylene) present having 6 or fewer carbon atoms;
Q is a covalent bond or an organic linking group selected from a sulfonamido group, a carboxamido group, a carboxyl group, or a sulfonyl group, preferably Q is a covalent bond,
m is 1 to 22, preferably 4 to 12, and
Z is a hydroxyl, thiol, or a primary or secondary amino group, preferably z is a hydroxyl group.

R_{f}-Q-(CH₂)ₘZ may comprise fluorochemical monoalcohols including the following:
R_{f}SO₂N(CH₃)CH₂CH₂OH, R_{f}SO₂N(H)(CH₂)₂OH, R_{f}SO₂N(CH₃)(CH₂)₄OH, R_{f}SO₂N(CH₃)(CH₂)₁₁OH, R_{f}SO₂N(C₂H₅)CH₂CH₂OH, R_{f}SO₂N(C₂H₅)(CH₂)₆OH, R_{f}SO₂N(C₂H₅)(CH₂)₁OH, R_{f}SO₂N(C₃H₇)CH₂OCH₂CH₂CH₂OH, R_{f}SO₂N(CH₂CH₂CH₃)CH₂CH₂OH, R_{f}SO₂N(C₄H₉)(CH₂)₄OH, R_{f}SO₂N(C₄H₉)CH₂CH₂OH, R_{f}CON(CH₃)CH₂CH₂OH, R_{f}CON(C₂H₅)CH₂CH₂OH, R_{f}CON(CH₃)(CH₂)₁₁OH, R_{f}CON(H)CH₂CH₂OH, R_{f}SO₂CH₂CH₂OH, R_{f}CO₂CH₂CH₂CH(CH₃)OH, R_{f}COOCH₂CH₂OH, R_{f}CH₂)₁₁N(C₂H₅)CH₂CH₂OH, R_{f}CH₂OH, R_{f}CH₂CH₂SO₂N(CH₃)CH₂CH₂OH, R_{f}CH₂)₂OH, R_{f}(CH₂)₂S(CH₂)₂OH, R_{f}CH₂CH₂CH₂OH, R_{f}CH₂)₄S(CH₂)₂OH, R_{f}CH₂)₂S(CH₂)₃OH, R_{f}(CH₂)₂SCH(CH₃)CH₂OH, R_{f}CH₂)₄SCH(CH₃)CH₂OH, R_{f}CH₂CH(CH₃)S(CH₂)₂OH, R_{f}(CH₂)₂S(CH₂)₁₁OH, R_{f}CH₂)₂S(CH₂)₃O(CH₂)₂OH, R_{f}CH₂)₃O(CH₂)₂OH, R_{f}(CH₂)₃SCH(CH₃)CH₂OH, R_{f}CH₂)₄OH, R_{f}CH₂)₁₁OH, R_{f}(CH₂)₂₂OH, and the like, and mixtures thereof, wherein R_{f} is a perfluoroalkyl group having 1 to 22 carbon atoms, or a perfluoroheteroalkyl group having 3 to about 50 carbon atoms with all perfluorocarbon chains present having 6 or fewer carbon atoms, and m is 1 to 22. If desired, rather than using such alcohols, the analogous thiols or amines can be used.

Specific fluorochemical monoalcohols including the following:
CF₃(CF₂)₃SO₂N(CH₃)CH(CH₃)CH₂OH, CF₃(CF₂)₃SO₂N(CH₃)CH₂CH(CH₃)OH, C₄F₉SO₂N(CH₃)(CH₂)₄OH, C₆F₁₃SO₂N(CH₃)(CH₂)₄OH, CF₃(CF₂)₃SO₂N(C₂H₅)CH₂CH₂OH, C₆F₁₃SO₂N(C₂H₅)CH₂CH₂OH, C₃F₇CONHCH₂CH₂OH, C₂F₅O(C₂F₄O)₃CF₂CONHC₂H₄OH, CF₃O(CF(CF₃)CF₂O)₁₋₃₆CF(CF₃)CH₂OH, C₂F₅O(CF(CF₃)CF₂O)₁₋₃₆CF(CF₃)CH₂OH, C₃F₇O(CF(CF₃)CF₂O)₁₋₃₆CF(CF₃)CH₂OH, C₄F₉O(CF(CF₃)CF₂O)₁₋₃₆CF(CF₃)CH₂OH, C₃F₇O(CF(CF₃)CF₂O)₁₂CF(CF₃)CH₂OH, CF₃O(CF₂CF₂O)₁₋₃₆CF₂CH₂OH, C₂F₅O(CF₂CF₂O)₁₋₃₆CF₂ CH₂OH, C₃F₇O(CF₂CF₂O)₁₋₃₆CF₂CH₂OH, C₄F₉O(CF₂CF₂O)₁₋₃₆CF₂CH₂OH, n-C₄F₉OC₂F₄OCF₂CH₂CH₂CH₂OH, CF₃O(CF₂CF₂O)₁₁CF₂CH₂OH, C₅F₁₁COOCH₂CH₂OH, C₃F₇CH₂OH, perfluoro(cyclohexyl)methanol, C₄F₉CH₂CH₂OH, CF₃(CF₂)₅CH₂CH₂OH, CF₃(CF₂)₅CH₂CH₂SO₂N(CH₃)CH₂CH₂OH, CF₃(CF₂)₃CH₂CH₂SO₂N(CH₃)CH₂CH₂OH, C₄F₉(CH₂)₂S(CH₂)₂OH, C₄F₉(CH₂)₄OH, C₄F₉(CH₂)₁₁OH, C₈F₁₇(CH₂)₁₁OH, CF₃(CF₂)₃SO₂N(CH₃)CH₂CH₂OH, and C₄F₉(CH₂)₂₂OH.

Preferred fluorine-containing monoalcohols include 2-(N-methylperfluorobutanesulfonamido)ethanol; 2-(N-ethylperfluorobutanesulfonamido)ethanol; 2-(N-methylperfluorobutanesulfonamido)propanol; N-methyl-N-(4-hydroxybutyl)perfluorohexanesulfonamide; 1,1,2,2-tetrahydroperfluorooctanol; 1,1-dihydroperfluorooctanol; C₆F₁₃CF(CF₃)CO₂C₂H₄CH(CH₃)OH; n-C₆F₁₃CF(CF₃)CON(H)CH₂CH₂OH; C₄F₉OC₂F₄OCF₂CH₂OCH₂CH₂OH; C₃F₇ CON(H)CH₂CH₂OH; 1,1,2,2,3,3-hexahydroperfluorodecanol; C₃F₇O(CF(CF₃)CF₂O)₁₋₃₆CF(CF₃)CH₂OH; CF₃O(CF₂CF₂O)₁₋₃₆CF₂CH₂OH; C₄F₉(CH₂)₄OH, C₄F₉(CH₂)₁₁OH, C₈F₁₇(CH₂)₁₁OH, CF₃(CF₂)₃SO₂N(CH₃)CH₂CH₂OH, and C₄F₉(CH₂)₂₂OH, C₄F₉(CH₂)₆OH and the like; and mixtures thereof.

With respect to Formula IV, perfluorinated dihydroalcohols of the general formula R_{f}—CH₂—OH may be prepared by reduction of the corresponding perfluorinated acyl fluoride or ester. Higher fluorinated alcohols may be prepared by the reaction of a perfluoropolyether iodide or perfluoroalkyl iodide (prepared using the procedure described in J. L. Howell et al., J. Fluorine Chem., vol.125, (2004), p. 1513) with an α-unsaturated, ω-hydroxy compound using a free radical catalyst such as benzoyl peroxide or AIBN. The obtained iodo-alcohol can be reduced to remove the secondary iodide, such as with zinc/acetic acid. The hydroxyl group may be converted to other nucleophilic functional groups “Z” by means known in the art.

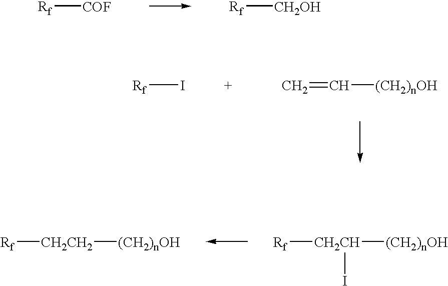

Perfluoropolyether compounds can be obtained by oligomerization of hexafluoropropylene oxide (HFPO) which results in a perfluoropolyether carbonyl fluoride. This carbonyl fluoride may be converted into an alcohol, thiol or amine by reactions well known to those skilled in the art.

The coupling agent may be selected from carbodiimides, and N,N′-carbonyldiimidazole. Preferred coupling agents are dialkyl and diaryl carbodiimides, such as N,N′-dicyclohexylcarbodiimide, N,N′-diisopropylcarbodiimide N,N′-di-tert-butylcarbodiimide, and N-ethyl-N′-(3-dimethylaminopropyl)carbodiimide. The coupling agent may be used in amounts of one to two molar equivalents, relative to the amount of fluorochemical monofunctional compound. In some embodiments the carbonyldiimidazole may be preferred, due to the ease of removing the byproduct imidazole from the product mixture by aqueous extraction.

Polymer bound coupling agents may be advantageously used, such as polymer bound N-benzyl-N′-cyclohexylcarbodiimide, described in Desai, M. C. et al. Tetrahedron Lett. 1993, 34, 7685; Buckman, B. O. et al. ibid. 1998, 39, 1487; Weinshenker, G. et al. Org. Synth. Coll. Vol. VI, 951, 1988; and Guan, Y. et al. J. Comb. Chem. 2000, 2 ,297.

The reaction mixture may further include a non-nucleophilic base for the coupling reaction. The term “non-nucleophilic base” means a base which does not undergo an irreversible reaction with the carboxylic acid group or the coupling agent. This reaction, when it occurs will reduce the yields of the desired benzotriazole. The non-nucleophilic base may be an organic or inorganic base, but is preferably an organic aprotic base. Examples of suitable organic non-nucleophilic bases include alkylamines, for example triethyl amine, trimethyl amine, tripropyl amine and diisopropylethyl amine, pyridines, alkyl pyridines and dialkylaminopyridines, alkyl piperidines, dialkyl piperazines, N-alkyl pyrrolidines, N-alkylpyrroles, N-alkylimidazoles, amidines, for example 1,5-diazabicyclo[4.3.0]non-5-ene (DBN) or 1,8-diazabicyclo[5.4.0]undec-7-ene (DBU), or guanidines. It will be understood that if the monofunctional fluorochemical compound is an amine, an additional amine catalyst is not required. The amine catalyst, if present, is generally used in amounts of 0.1 to 15 molar percent, preferably 5 to 10%, relative to the amount of coupling agent.

The fluorochemical benzotriazoles may be made according to the following step-wise synthesis. As one skilled in the art would understand, the order of the steps is non-limiting and can be modified so as to produce a desired chemical composition. In the synthesis, the carboxybenzotriazole, the monofunctional fluorochemical compound, and the coupling agent are mixed, preferably dissolved, together under dry conditions, preferably in a solvent. The resulting mixture or solution may be heated at approximately 20 to 100° C., preferably 30 to 75° C., with mixing for a period of time sufficient to effect the coupling reaction.

As the fluorochemical monofunctional compound is generally the more expensive starting material, a molar excess of the carboxybenzotriazole (as a function of carboxyl molar equivalents) may be used. In other embodiments, the molar amounts of fluorochemical monofunctional compound and carboxybenzotriazole can vary from 2:1 to 1:2, and is preferably about 1:1, relative to carboxyl molar equivalents. Excess carboxybenzotriazole may be recovered from the product mixture as the carboxybenzotriazole/coupling agent adduct. With judicious selection of the solvent, the fluorochemical benzotriazole product remains soluble in the solvent, while the carboxybenzotriazole/coupling agent adduct and the urea byproduct of the coupling agent substantially precipitate from solution. If desired, the soluble fluorochemical benzotriazole product may be recovered from the solution and recrystallized using techniques known in the art.

The solvent should be selected to be non-reactive toward the components of the mixture, and to provide sufficient solubility thereto. Preferably the solvent is selected so that the starting materials are soluble in the solvent, but the byproducts of the coupling reaction are not. Suitable solvents include esters, glycol ethers, amides, ketones, hydrocarbons, chlorohydrocarbons, chlorocarbons, and mixtures thereof. Mixtures of solvents may be used. Any solvent used should be anhydrous, (e.g., less than 0.1% water) to avoid competing hydrolysis of the coupling agent.

Polar aprotic solvents are preferred. Examples of suitable aprotic liquids include linear ethers such as diethylether, diethylene glycol dimethyl ether, and 1,2-dimethoxyethane; cyclic ethers such as tetrahydrofuran, 2-methyltetrahydrofuran, dioxane, dioxolane, and 4-methyldioxolane; nitrites such as acetonitrile and benzonitrile; nitro compounds such as nitromethane or nitrobenzene; amides such as N,N-dimethylformamide, N,N-diethylformamide, and N-methylpyrrolidinone; sulfoxides such as dimethyl sulfoxide; sulfones such as dimethylsulfone, tetramethylene sulfone, and other sulfolanes; oxazolidinones such as N-methyl-2-oxazolidinone and mixtures thereof.

### EXAMPLES

These examples are merely for illustrative purposes only and are not meant to be limiting on the scope of the appended claims. All parts, percentages, ratios, etc. in the examples and the rest of the specification are by weight, unless noted otherwise. Solvents and other reagents used were obtained from Sigma-Aldrich Chemical Company; Milwaukee, Wisconsin unless otherwise noted.

### Example 1

### Synthesis of 12,12,13,13,14,14,15,15,15-Nonafluoro-1-pentadecyl Benzotriazole-5-carboxylate [C₄F₉—(CH₂)₁₁—O₂C—C₆H₄N₃].

To a solution of 199.7 grams (0.577 mol) of perfluorobutyl iodide and 93.7 grams (0.550 mol) of 10-undecen-1-ol in a mixture of 700 milliliters of acetonitrile and 300 milliliters of water was added a mixture of 53.8 grams (0.640 mol) of NaHCO₃ and 106.2 grams (0.610 mol) of Na₂S₂O₄ in small portions with stirring. The reaction mixture was stirred at room temperature overnight and acidified with 1 N hydrochloric acid. The mixture was extracted with diethyl ether, and the combined organic phases were washed with saturated aqueous NaHCO₃ and saturated aqueous NaCl and dried over MgSO₄. Concentration afforded 234.4 grams of crude 12,12,13,13,14,14,15,15,15-nonafluoro-10-iodo-1-pentadecanol as a viscous, light amber liquid, which was used in the next step without further purification.

To a slurry of 130.0 grams (1.989 mol) of Zn powder in 500 milliliters of ethanol was added 5.0 grams of acetic acid. A solution of the crude iodide prepared above in 100 milliliters of ethanol was added dropwise with stirring over 1 hour, and the reaction mixture was heated at 50° C. for 4 hours. The mixture was filtered, the filtrate was concentrated to a viscous, light yellow liquid, and bulb-to-bulb distillation in several portions provided 97.3 grams (45% from 10-undecen-1-ol) of 12,12,13,13,14,14,15,15,15-nonafluoro-1-pentadecanol as a colorless solid, bp 160-200° C. at 0.05 mm.

To a mixture of 9.41 grams (24 mmol) of 12,12,13,13,14,14,15,15,15-nonafluoro-1-pentadecanol and 8.16 grams (50 mmol) of benzotriazole-5-carboxylic acid in 200 milliliters of a 9:1 mixture of tetrahydrofuran and dimethyl formamide were added 10.32 grams (50 mmol) of N,N-dicyclohexylcarbodiimide and 0.61 grams (5 mmol) of 4-(dimethylamino)pyridine, and the resultant mixture was heated at 70° C. for 48 hours. The mixture was filtered, and the filtrate was concentrated to a dark semisolid. The crude product was slurried in 200 milliliters of ethyl acetate, the mixture was filtered, and the filtrate was concentrated to a tan solid. Two recrystallizations from a 9:1 mixture of methanol and water afforded 9.26 grams (74%) of tan crystals, mp 99-102° C. The ¹H and ¹³C NMR spectra of the final product and all intermediates were consistent with the structures of the target compounds.

### Example 2

### Synthesis of 12,12,13,13,14,14,15,15,16,16,17,17,18,18,19,19,19-Heptadecafluoro-1-nonadecyl Benzotriazole-5-carboxylate [C₈F₁₇—(CH₂)₁₁—O₂C—C₆H₄N₃].

To a solution of 41.10 grams (75 mmol) of perfluorooctyl iodide and 11.92 grams (70 mmol) of 10-undecen-1-ol in a mixture of 100 milliliters of acetonitrile and 40 milliliters of water was added a mixture of 6.89 grams (82 mmol) of NaHCO₃ and 13.58 grams (78 mmol) of Na₂S₂O₄ in small portions with stirring. The reaction mixture was stirred at room temperature overnight and acidified with 1 N hydrochloric acid. The mixture was extracted with diethyl ether, and the combined organic phases were washed with saturated aqueous NaHCO₃ and saturated aqueous NaCl and dried over MgSO₄. Concentration afforded 43.2 grams of 12,12,13,13,14,14,15,15,16,16,17,17,18,18,19,19,19-heptadecafluoro-10-iodo-1-nonadecanol as a white solid, which was used in the next step without further purification.

A solution of the crude iodide prepared above in 50 milliliters of ethanol was filtered to remove a small amount of insoluble material, and the filtrate was added dropwise with stirring to a slurry of 19.6 grams (300 mmol) of Zn powder in 150 milliliters of ethanol containing 4.0 grams of acetic acid. The reaction mixture was heated at 50° C. for 4 hours. The mixture was filtered, and concentration of the filtrate gave approximately 45 grams of a soft white solid. A 10.0 grams portion of this material was triturated with hexanes, and filtration recovered 7.0 grams of a white solid. This material was stirred in 75 milliliters of water for 1 h to remove some acetic acid remaining in the product from the Zn reduction, and filtration yielded 4. 1 grams (50 % from 10-undecen-1-ol) of 12,12,13,13,14,14,15,15,16,16,17,17,18,18,19,19,19-heptadecafluoro-1-nonadecanol as a white solid, which was used in the next step without further purification.

To a mixture of 3.00 grams (5.1 mmol) of 12,12,13,13,14,14,15,15,16,16,17,17,18,18,19,19,19-heptadecafluoro-1-nonadecanol and 2.45 grams (15.0 mmol) of benzotriazole-5-carboxylic acid in 50 milliliters of a 9:1 mixture of tetrahydrofuran and dimethyl formamide were added 3.09 grams (15.0 mmol) of N,N-dicyclohexylcarbodiimide and 0.18 grams (1.5 mmol) of 4-(dimethylamino)pyridine, and the resultant mixture was heated at 70° C. for 48 h. The mixture was filtered, and the filtrate was concentrated to a dark tan solid. The crude product was slurried in 50 milliliters of tetrahydrofuran, the mixture was filtered, and the filtrate was concentrated to a tan solid. Two recrystallizations from ethanol provided 1.88 grams (50%) of tan crystals, mp 130-133° C. The ¹H and ¹³C NMR spectra of the final product and all intermediates were consistent with the structures of the target compounds.

### Example 3

### Synthesis of 23,23,24,24,25,25,26,26,26-Nonafluoro-1-hexacosyl Benzotriazole-5-carboxylate [C₄F₉—(CH₂)₂₂—O₂C—C₆H₄N₃].

A mixture of 4.00 grams of 21-docosen-1-ol, 10.00 grams (28.9 mmol) of perfluorobutyl iodide, and 0.O10 grams (0.6 mmol) of azobis(isobutyronitrile) was heated at 70° C. for 18 hours. Separation of volatiles under reduced pressure left 23,23,24,24,25,25,26,26,26-nonafluoro-21-iodo-1-hexacosanol as a light tan solid, which was used in the next step without further purification.

To a slurry of 5.00 (76 mmol) Zn powder in 500 milliliters of ethanol was added 10 drops of acetic acid. A solution of the crude iodide prepared above in 20 milliliters of ethanol was added dropwise with stirring, and the reaction mixture was heated at 50° C. for 3 hours. The mixture was filtered, the filtrate was concentrated to a 3:2 mixture of 23,23,24,24,25,25,26,26,26-nonafluoro-1-hexacosanol and 23,23,24,24,25,25,26,26,26-nonafluoro-21-hexacosen-1-ol as a white solid. Recrystallization from heptane yielded a 2:1 mixture of the same two compounds. Flash chromatography of this mixture on silica with a 1:1 mixture of hexanes and diethyl ether provided a 9:1 mixture of these two compounds. To a solution of this mixture in 100 milliliters of a 1:1 mixture of hexanes and ethanol was added 100 milligrams of 5% Pd/C, and this mixture was hydrogenated at 50 psi of hydrogen using a Parr hydrogenator. Filtration and concentration gave 1.69 grams (25% from 21-docosen-1-ol) of 23,23,24,24,25,25,26,26,26-nonafluoro-1-hexacosanol as a white solid, mp 69-71° C.

To a mixture of 0.50 grams (0.9 mmol) of 23,23,24,24,25,25,26,26,26-nonafluoro-1-hexacosanol and 0.49 grams (3.0 mmol) of benzotriazole-5-carboxylic acid in 10 milliliters of a 9:1 mixture of tetrahydrofuran and dimethyl formamide were added 0.62 grams (3.0 mmol) of N,N-dicyclohexylcarbodiimide and 0.04 grams (0.3 mmol) of 4-(dimethylamino)pyridine, and the resultant mixture was heated at 70° C. for 48 hours. The mixture was filtered, and the filtrate was concentrated to a dark tan semisolid. The crude product was slurried in 30 milliliters of tetrahydrofuran, the mixture was filtered, and the filtrate was concentrated to a tan solid. Recrystallization from methanol provided 0.50 grams (79%) of tan crystals, mp 106-108° C. The ¹H and ¹³C NMR spectra of the final product and all intermediates were consistent with the structures of the target compounds.

### Example 4

### Synthesis of 5,5,6,6,7,7,8,8,8-Nonafluoro-1-octyl Benzotriazole-5-carboxylate [C₄F₉—(CH₂)₄—O₂C—C₆H₄N₃].

To a solution of 190.3 grams (0.550 mol) of perfluorobutyl iodide and 36.1 grams (0.500 mol) of 3-buten-1-ol in a mixture of 560 milliliters of acetonitrile and 240 milliliters of water was added a mixture of 48.3 grams (0.575 mol) of NaHCO₃ and 95.8 grams (0.550 mol) of Na₂S₂O₄ in small portions with stirring. The reaction mixture was stirred at room temperature overnight and acidified with 200 milliliters of 1 N hydrochloric acid and diluted with 400 milliliters of water. The mixture was extracted with diethyl ether, and the combined organic phases were washed with saturated aqueous NaHCO₃ and saturated aqueous NaCl and dried over MgSO₄. Concentration afforded 38.5 grams of crude 5,5,6,6,7,7,8,8,8-nonafluoro-3-iodo-1-octanol as a clear orange liquid, which was used in the next step without further purification.

To a slurry of 29.4 grams (450 mmol) of Zn powder in 400 milliliters of ethanol was added a solution of the crude iodide prepared above in 50 milliliters of ethanol, and the reaction mixture was heated at 50° C. for 4 hours. The mixture was filtered, the filtrate was concentrated to a light orange liquid, and bulb-to-bulb distillation provided 19.6 grams (13% from 3-buten-1-ol) of 5,5,6,6,7,7,8,8,8-nonafluoro-1-octanol as a colorless liquid, bp 95-105° C. at 0.1 mm.

To a mixture of 8.76 grams (30 mmol) of 5,5,6,6,7,7,8,8,8-nonafluoro-1-octanol and 9.79 grams (60 mmol) of benzotriazole-5-carboxylic acid in 200 milliliters of a 9:1 mixture of tetrahydrofuran and dimethyl formamide were added 12.38 grams (60 mmol) of N,N-dicyclohexylcarbodiimide and 0.73 grams (6 mmol) of 4-(dimethylamino)pyridine, and the resultant mixture was heated at 70° C. for 48 hours. The mixture was filtered, and the filtrate was concentrated to a tan solid. The crude product was slurried in 250 milliliters of ethyl acetate, the mixture was filtered, and the filtrate was concentrated to a tan solid. Flash chromatography on silica with ethyl acetate followed by recrystallization from a 95:5 mixture of heptane and 2-propanol afforded 6.77 grams (52%) of tan crystals, mp 98-101° C. The ¹H and ¹³C NMR spectra of the final product and all intermediates were consistent with the structures of the target compounds.

### Synthesis Example 1

### Preparation of 7,7,8,8,9,9,10,10,10-Nonafluoro-5-iodo-decan-1-ol

A solution of sodium bicarbonate (3.15 grams, 37.5 mmol) dissolved in 50 milliliters of water was added to a flask. Nonafluoro-1-iodobutane (15.5 milliliters, 90 mmol) and 5-hexen-1-ol (9.0 milliliters, 75 mmol) were added and the reaction mixture was stirred for a couple of minutes at room temperature. The reaction mixture was placed in a sonic bath and powdered sodium dithionite (4.35 grams, 25 mmol) was added over a period of ten minutes. Sonication was continued for 15 minutes and the reaction mixture (two clear liquid phases) was stirred for an additional hour. The mixture was transferred to a separatory funnel and the bottom layer (organic) was isolated. The organic portion was washed successively with H₂0 and brine and concentrated to give an oil. NMR of the crude material showed about 80% conversion with some 5-hexen-1-ol remaining. The 5-hexen-1-ol was removed by rotary evaporation at 80° C. under high vacuum to yield 7,7,8,8,9,9,10,10,10-nonafluoro-5-iodo-decan-1-ol (22.9 grams) as a colorless liquid.

### Synthesis Example 2

### Preparation of 7,7,8,8,9,9,10,10,10-Nonafluoro-decan-1-ol

A solution of 7,7,8,8,9,9,10,10,10-nonafluoro-5-iodo-decan-1-ol (19.1 grams, 42.8 mmol) dissolved in 150 milliliters of methanol was placed in a 500-milliliters Parr pressure bottle. Sodium bicarbonate (8.5 grams, 101 mmol) was added followed by 10% palladium on charcoal (1.91 grams) and the mixture was shaken under an atmosphere of H₂ (50 PSI) for 4 days. The reaction mixture was filtered through Celite and the filtrate was concentrated under reduced pressure to give colorless oil containing a white precipitate. The mixture was dissolved in 50 milliliters of ethyl acetate and the solution was washed successively with saturated NaHCO₃ solution, H₂O and brine. The organic portion was dried over Na₂SO₄, filtered and concentrated under reduced pressure to give a colorless liquid. Distillation (98-100° C., 15 mmHg) gave 7,7,8,8,9,9,10,10,10-nonafluoro-decan-1-ol (14.7 grams) as a colorless liquid.

### Synthesis Example 3

### Alternative Preparation of 7,7,8,8,9,9,10,10,10-Nonafluoro-decan-1-ol

### Step 1: Preparation of Acetic acid 7,7,8,8,9,9,10,10,10-nonafluoro-5-iodo-decyl ester

5-Hexene-1-ol acetate (10.0 milliliters, 63 mmol) and nonafluoro-1-iodobutane (17.2 milliliters, 100 mmol) were combined in a round-bottomed flask. The mixture was treated with AIBN (328 milligrams, 2.00 mmol) and then heated to 70° C. overnight under an atmosphere of N₂. The reaction mixture was concentrated under reduced pressure to remove the excess nonafluoro-1-iodobutane. The resulting acetic acid 7,7,8,8,9,9,10,10,10-nonafluoro-5-iodo-decyl ester (29.4 grams) was sufficiently pure to use in the subsequent reduction (Step 2).

### Step 2: Preparation of 7,7,8,8,9,9,10,10,10-Nonafluoro-decan-1-ol

Acetic acid 7,7,8,8,9,9, 10,10,10-nonafluoro-5-iodo-decyl ester (29.4 grams, 60.2 mmol) was dissolved in 50 milliliters of ethanol and treated with zinc dust (11.7 grams, 180 mmol). A 3.0 Molar solution of HCl in ethanol was then added dropwise (with rapid gas evolution) over a period of 90 minutes. Gas evolution was still observed for about an hour after the addition was complete. Stirring was continued at ambient temp overnight after which time most of the zinc dust had been consumed. The reaction mixture was then heated to reflux for several hours to complete the hydrolysis of the ester. The reaction mixture was concentrated under reduced pressure to remove the ethanol and the resulting oil was dissolved in 200 milliliters of ether and washed successively with H₂O (2×) and brine. The organic layer was dried over MgSO₄, filtered and concentrated under reduced pressure. The resulting liquid was subjected to vacuum distillation. The first 0.5 milliliters (bp 96-98° C., 15 mmHg) was discarded. The fraction that distilled at 98-100° C. was collected to give 7,7,8,8,9,9,10,10,10-nonafluoro-decan-1-ol (13.9 grams) as a colorless liquid.

### Example 5

### Synthesis of 1H-Benzotriazole-5-carboxylic acid 7,7,8,8,9,9,10,10,10-nonafluoro-decyl ester

Benzotriazole-5-carboxylic acid (3.75 grams, 23.0 mmol) was dissolved in 15 milliliters of anhydrous DMF and stirred under N₂. N,N′-carbonyldiimidazole (3.75 grams, 23.0 mmol) was added to the reaction and the mixture was stirred until gas evolution ceased (about 5 minutes). 7,7,8,8,9,9,10,10,10-nonafluorodecanol (5.45 milliliters, 23.0 mmol) was added and the mixture was heated to 80° C. for 2 days. The reaction mixture was cooled and concentrated under reduced pressure to give a dark brown syrup. The syrup was poured into 150 milliliters of ice water followed by rapid stirring. After 15 minutes, the mixture was filtered to give a pasty, brown sludge that was rinsed with H₂O and dried somewhat with suction. The brown sludge was then transferred to a flask and dissolved in ethyl acetate. The solvent was evaporated to give an oil that started to solidify upon standing. The material was dissolved in about 10 milliliters of ethyl acetate and applied to a 6×3 cm pad of SiO₂. The pad was eluted with about 200 milliliters of ethyl acetate and the filtrate was concentrated give 11 grams of brown solid. The brown solid was dissolved in 100 milliliters of hot ethanol and treated with 5 grams of charcoal. The hot solution was filtered through a pad of Celite and concentrated to give a light brown solid. Crystallization from 25 milliliters of 10% ethyl acetate/hexanes gave 3.6 grams of the desired product and an off-white powder, m.p. 86.5-90.5° C. ¹H NMR confirmed the predicted structure. Anal. Calcd for C₁₇H₁₆F₉N₃O₂: C, 43.88; H, 3.47; N, 9.03. Found: C, 43.87, H, 3.44, N, 8.93.

1. A method of preparing fluorochemical benzotriazoles comprising the step of contacting a fluorochemical monofunctional compound with a carboxybenzotriazole in the presence of a coupling agent, and optionally an amine catalyst.

2. The method of claim 1 where the fluorochemical benzotriazole is of the 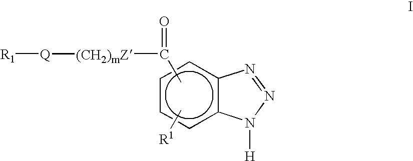
formula:
wherein R_{f} is a perfluoroalkyl or a perfluoroheteroalkyl group, Q is a covalent bond or an organic linking group selected from a sulfonamido group, a carboxamido group, a carboxyl group, or a sulfonyl group, Z′ is —O—, —S—, or —NR—, where R⁵ is H or C₁-C₄ alkyl, R¹ is H, a C₁-C₆ alkyl, or R_{f}-Q-(CH₂)ₘ-Z′-C(O)—, and m is at least 1.

3. The method of claim 1 wherein the fluorochemical monofunctional compound is of the formula R_{f}-Q-(CH₂)ₘZ
wherein:
R_{f} is a perfluoroalkyl group having 1 to 22 carbon atoms, or a perfluoroheteroalkyl group having 3 to about 50 carbon atoms with all perfluorocarbon chains present having 6 or fewer carbon atoms;
Q is a covalent bond or an organic linking group selected from a sulfonamido group, a carboxamido group, a carboxyl group, or a sulfonyl group,
m is 1 to 22, and
Z is a hydroxyl, thiol, or a primary or secondary amino group.

4. The method of claim 1 wherein the carboxybenzotriazole is a 5-carboxybenzotriazole.

5. The method of claim 1 wherein the carboxybenzotriazole is a 5, 6-bis-carboxybenzotriazole.

6. The method of claim 1, wherein the coupling agent is selected from dialkyl and diaryl carbodiimides.

7. The method of claim 6 wherein the carbodiimide coupling agent is selected from N,N′-dicyclohexylcarbodiimide, N,N′-diisopropylcarbodiimide N,N′-di-tert-butylcarbodiimide.

8. The method of claim 1 wherein the carbodiimide coupling agent is a polymer-supported carbodiimide coupling agent.

9. The method of claim 1 where the molar ratio of fluorochemical monofunctional compound to the carboxybenzotriazole is 2:1 to 1:2.

10. The method of claim 1 wherein the R_{f} group is of the formula CₙF₂ₙ₊₁, where n is 3 to 5.

11. The method of claim 1 wherein m is 4 to 12.

12. The method of claim 1 wherein the partially fluorinated alcohol and the carboxybenzotriazole comprise a solvent solution.

13. The method of claim 1 wherein the coupling agent is present in amounts of one to two molar equivalents, relative to the amount of fluorochemical monofunctional compound.

14. The method of claim 1 further comprising the step of recovering the fluorinated benzotriazole compound.

15. The method of claim 1 comprising providing a mixture of a fluorinated alcohol, a carboxybenzotriazole, a coupling agent, and an amine catalyst in a solvent or mixture of solvents.
